(19) United States
(12) Patent Application Publication
(10) Pub. No.: US 2025/0025512 A1
(43) Pub. Date: Jan. 23, 2025

(54) HUMAN INDUCED PLURIPOTENT STEM CELLS-DIFFERENTIATED CARDIOMYOCYTES, METHODS OF PRODUCING THE SAME, AND USES THEREOF IN TREATING CARDIAC DISEASES

(71) Applicant: NATIONAL CHENG KUNG UNIVERSITY, Tainan City (TW)

(72) Inventors: Yen-Wen LIU, Tainan City (TW); Yan-Shen SHAN, Tainan City (TW)

(73) Assignee: NATIONAL CHENG KUNG UNIVERSITY, Tainan City (TW)

(21) Appl. No.: 18/353,885

(22) Filed: Jul. 18, 2023

(51) Int. Cl. A61K 35/34; A61P 9/10; C12N 5/077; C12N 9/22; C12N 15/11; C12N 15/90

(52) U.S. Cl. CPC A61K 35/34; A61P 9/10; C12N 5/0657; C12N 9/22; C12N 15/11; C12N 15/907; C12N 2310/20; C12N 2500/32; C12N 2500/38; C12N 2501/30; C12N 2501/415; C12N 2506/45; C12N 2800/80

(57) ABSTRACT

Disclosed herein is an isolated population of cardiomyocytes differentiated from human induced pluripotent stem cells (hiPSCs), wherein the isolated population of cardiomyocytes comprises genomic deletions in HCN1 and HCN4 genes. According to some embodiments of the present disclosure, the isolated population of cardiomyocytes further comprises genomic deletions in HCN2 and HCN3 genes. Also disclosed herein are methods of producing the isolated population of cardiomyocytes, and methods of treating subjects suffering from cardiac diseases by administering the isolated population of cardiomyocytes to the subjects.

The present application is being filed along with a Sequence Listing XML in electronic format. The Sequence Listing XML is provided as a XML file entitled AJ23014N_Sequence_Listing, created Jul. 5, 2023, which is 32 Kb in size. The information in the electronic format of the Sequence Listing XML is incorporated herein by reference in its entirety.

### BACKGROUND OF THE INVENTION

### 1. Field of the Invention

The present disclosure in general relates to the field of disease treatment. More particularly, the present disclosure relates to a population of human induced pluripotent stem cells (hiPSCs)-differentiated cardiomyocytes and uses thereof in treating cardiac diseases.

### 2. Description of Related Art

Cardiac disease is one of leading causes of death globally. It refers to several types of heart conditions, including coronary heart disease, myocardial infarction, cardiac arrhythmia, myocardial ischemia, and cardiac failure. According to the Centers for Disease Control and Prevention (CDC), about 695,000 people in the United States died from cardiac disease in 2021, i.e., one in every five deaths. A major reason of the high mortality rate of cardiac disease is that the heart is one of the least regenerative organs in the body; when damage occurs to the myocardium, cardiomyocytes (native cardiac muscle cells), are replaced with fibrotic scar tissue, a pathophysiologic process known as “cardiac fibrosis” that leads to cardiac dysfunction and heart failure subsequently.

Over the past 20 years, there have been extensive efforts to induce the heart to heal by muscle regeneration rather than scarring. It is reported that cardiomyocytes can be derived from human pluripotent stem cells (hPSCs), such as human embryonic stem cell (hESC) or human induced pluripotent stem cells (hiPSC). The hPSC-derived cardiomyocytes survive after transplantation and form new, maturing myocardium in animal models of myocardial infarction. However, some animals experienced graft-associated ventricular arrhythmias, including ventricular tachycardia and accelerated idioventricular rhythm (AIVR).

In view of the forging, there exists in the related art a need for an improved method for treating cardiac disease without causing adverse effect, e.g., ventricular arrhythmias.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to an isolated population of cardiomyocytes. According to the embodiments of the present disclosure, the isolated population of cardiomyocytes is differentiated from human induced pluripotent stem cells (hiPSCs).

According to some embodiments, the isolated population of cardiomyocytes comprises genomic deletions in HCN1 and HCN4 genes, and accordingly, does not express hyperpolarization-activated cyclic nucleotide gated potassium channel (HCN) 1 and HCN4. According to some embodiments, in addition to HCN1 and HCN4 genes, the isolated population of cardiomyocytes further comprises genomic deletions in HCN2 and HCN3 genes; in the embodiments, the isolated population of cardiomyocytes does not express HCN1, HCN2, HCN3 and HCN4.

Also disclosed herein is a method of producing the isolated population of cardiomyocytes of the present disclosure. The method comprises,
(a) making deletions in the HCN1 and HCN4 genes of the hiPSCs; and
(b) incubating the hiPSCs of step (a) in a differentiation medium to induce differentiation of the hiPSCs into the population of cardiomyocytes.

According to certain embodiments, the method further comprises making deletions in the HCN2 and HCN3 genes of the hiPSCs prior to step (b).

Another aspect of the present disclosure pertains to a method of treating a cardiac disease in a subject. The method comprises administering to the subject an effective amount of the isolated population of cardiomyocytes of the present disclosure, so as to ameliorate or alleviate the symptoms of the cardiac disease.

According to some embodiments, the isolated population of cardiomyocytes is intramyocardially administered to the subject. In some exemplary embodiments, the isolated population of cardiomyocytes is administered to the left ventricle of the heart of the subject.

Depending on desired purposes, the cardiac disease may be myocardial infarction, cardiac arrhythmia, myocardial ischemia, cardiac failure, angina pectoris, or a combination thereof.

The subject is a mammal; preferably, a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a histogram depicting the effect of the present HCN-deleted hiPSC-CMs on inhibiting post-transplant ventricular arrhythmias according to Example 2 of the present disclosure. HCN1-4-positive hiPSC-CMs: cardiomyocytes derived from hiPSC expressing HCN1, HCN2, HCN3 and HCN4 genes; HCN1-4-deleted hiPSC-CMs: cardiomyocytes derived from the HCN1⁻/HCN2⁻/HCN3⁻/HCN4⁻ hiPSCs. Pre-MI: ventricular arrhythmia frequency of animals prior to myocardial infarction (ischemia-reperfusion, I-R) surgery; Post-MI, Pre-cell transplantation: ventricular arrhythmia frequency of animals after myocardial infarction surgery and prior to hiPSC-CM transplantation; Post-cell transplantation: ventricular arrhythmia frequency of animals after hiPSC-CM transplantation.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “cardiomyocytes” is given its ordinary meaning in the art and refers to sarcomere-containing striated muscle cells, which are found in the mammalian heart, as opposed to skeletal muscle cells. The term “cardiomyocyte” as used herein includes any cardiomyocyte subpopulation or cardiomyocyte subtype, e.g. atrial, ventricular and pacemaker cardiomyocytes.

As used herein, the term “human induced pluripotent stem cell” (hiPSC) is a type of pluripotent stem cell artificially derived from a non-pluripotent cell, typically an adult somatic cell, via inducing the expression of certain de-differentiation genes in the non-pluripotent cell, e.g., Oct3/4, Sox2, Klf4 and/or c-Myc genes. Human induced pluripotent stem cells are identical to human embryonic stem cells in the ability to form any adult cell, but are not derived from an embryo.

The term “delete” or “deletion” when used in reference to a gene refers to the partial or complete removal of nucleic acid sequence as it normally presents, or the partial or complete inactivation of nucleic acid sequence as it normally functions. Specifically, in the present disclosure, a deletion in a sequence means the removal of all or part of the sequence which may results in the complete or partial inactivation of the sequence. Deletions, or disruptions will render the gene or coding sequence “non-functional” within the meaning the present disclosure. According to the present disclosure, the expression of “deletion in HCN gene” and its grammatical equivalents (e.g., deletion in HCN1, HCN2, HCN3 or HCN4 gene) refer to a cardiomyocyte wherein a nucleic acid encoding all amino acids or a subset of the amino acids of HCN (e.g., HCN1, HCN2, HCN3 or HCN4) are not present. According to the present invention, if the cardiomyocyte comprising a deletion in the HCN gene has a residual nucleic acid encoding a subset of the amino acids of HCN, said residual nucleic acid is incapable of producing a functional gene product or the gene product is incapable of generating current in the heart.

The terms “administered”, “administering” and “administration” are used interchangeably herein to refer a mode of delivery. According to some embodiments, the present cardiomyocytes are intramyocardially injected to a subject in need thereof (e.g., a subject having or suspected of having a cardiac disease). In some preferred embodiments, the present cardiomyocytes are administered to the left ventricle of the heart of the subject.

As used herein, the terms “treating” and “treatment” are interchangeable, and encompass partially or completely preventing, ameliorating, mitigating and/or managing a symptom, a secondary disorder or a condition associated with cardiac disease. The term “treating” as used herein refers to application or administration of the present cardiomyocytes to a subject, who has a symptom, a secondary disorder or a condition associated with cardiac disease, with the purpose to partially or completely alleviate, ameliorate, relieve, delay onset of, inhibit progression of, reduce severity of, and/or reduce incidence of one or more symptoms, secondary disorders or features associated with cardiac disease. Symptoms, secondary disorders, and/or conditions associated with cardiac disease include, but are not limited to, chest pain, chest tightness, chest pressure, chest discomfort (e.g., angina pectoris), shortness of breath, dizziness, lightheadedness, fainting (syncope), fluttering in the chest, slow heartbeat (bradycardia), racing heartbeat (tachycardia), and fatigue. Treatment may be administered to a subject who exhibits only early signs of such symptoms, disorder, and/or condition for the purpose of decreasing the risk of developing the symptoms, secondary disorders, and/or conditions associated with cardiac disease. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed. Effective amount may be expressed, for example, in cell numbers of body weight (cells/Kg), cell numbers of body surface area (cells/m²), or cell numbers per subject (cells/subject). Persons having ordinary skills could calculate the human equivalent dose (HED) for the medicament (such as the present cardiomyocytes) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “subject” refers to a mammal including the human species that can be subjected to the isolated population of cardiomyocytes, pharmaceutical composition and/or method of the present invention. The term “subject” is intended to refer to both the male and female gender unless one gender is specifically indicated.

### II. DESCRIPTION OF THE INVENTION

The present disclosure is based, at least in part, on the unexpected discovery that the deletion of HCN genes, especially HCN1 and HCN4 genes, in hiPSC-derived cardiomyocytes (i.e., the cardiomyocytes differentiated from hiPSCs, hereinafter as “hiPSC-CMs) reduces the risk or tissue electrical disturbance and arrhythmia after transplantation into a subject. Accordingly, the present disclosure is directed to a population of hiPSC-CMs, and uses thereof in treating cardiac diseases. According to some embodiments of the present disclosure, the administration of the present hiPSC-CMs is useful in treating cardiac diseases without causing adverse effect, e.g., ventricular arrhythmias, in a subject.

The first aspect of the present disclosure is thus directed to a population of hiPSC-CMs. According to the embodiments of the present disclosure, the present population of hiPSC-CMs is characterized by having deletions in HCN1 and HCN4 genes, and accordingly, does not express HCN isoforms 1 and 4, i.e., HCN1 and HCN4. According to some preferred embodiments of the present disclosure, in addition to the deletions in HCN1 and HCN4 genes, the present population of hiPSC-CMs further comprises deletions in HCN2 and HCN3 genes; in these embodiments, the population of hiPSC-CMs does not express HCN isoforms 1, 2, 3 and 4, i.e., HCN1, HCN2, HCN3 and HCN4.

According to some exemplary embodiments of the present disclosure, the population of hiPSC-CMs is produced from the hiPSCs by a method comprising,
(a) making deletions in the HCN genes of the hiPSCs; and
(b) incubating the HCN-deleted hiPSCs of step (a) in a differentiation medium to induce differentiation of the HCN-deleted hiPSCs into the population of cardiomyocytes.

The hiPSCs for producing the present hiPSC-CMs may be obtained by any methods known in the art; for example, modifying the expression of transcription factors in human somatic cells so as to reprogram (di-differentiate) the somatic cells into pluripotent cells. Non-limiting examples of human somatic cells suitable to produce the present hiPSCs include, peripheral blood mononuclear cells (PBMCs), fibroblasts (e.g., epithelial fibroblasts from skin), keratinocytes (e.g., keratinocytes from hair follicles), mesenchymal stem cells (MSCs, e.g., mesenchymal stem cells from teeth or adipose tissue), epithelial cells (e.g., renal or gastric epithelial cells), hepatocytes, immune cells (e.g., T or B cells from peripheral blood), hematopoietic stem cells (HSCs), and bone marrow cells (BMCs). According to one exemplary embodiment, the hiPSC is derived from PBMCs.

Examples of transcription factors suitable for reprogramming somatic cells into pluripotent cells (e.g., the present hiPSCs) include, but are not limited to, octamer-binding transcription factor 3 (Oct3), Oct4, sex-determining region Y-box 1 (Sox1), Sox2, Sox3, Sox15, Sox18, Kruppel Like Factor (Klf1), Klf2, Klf4, Klf5, c-Myc, Nanog, LIN28, Glis1, and the combination thereof. According to some embodiments, human somatic cells are reprogramming into pluripotent cells by use of a four-factor reprogramming cocktail, known as “OSKM”, which include transcription factors Oct4, Sox2, KLF4 and c-Myc. According to certain embodiments, human somatic cells are reprogrammed by a six-factor reprogramming cocktail that includes transcription factors Oct4, Sox2, Klf4, c-Myc, Nanog and Lin28. Alternatively, one or more factors of the reprogramming cocktail OSKM (e.g., Klf4) may be in combination with other factors known to regulate the reprogramming process, for example, p53 inhibitor (e.g., p53 shRNA or SV40LT), miR-302b, miR-372, tranylcypromine, valproic acid (VPA), CHIR99021 (a GSK-3 inhibitor), E-616452 (an Alk5 inhibitor), or a combination thereof. As could be appreciated, the transcription factors used to reprogram the somatic cells into hiPSCs vary with the cells source and/or reprogramming method. A skilled artisan may select suitable transcription factors for the reprogramming process in accordance with intended purpose. According to some exemplary embodiment, transcription factors Oct4, Sox2, Klf4 and c-Myc are used to reprogram the somatic cells (e.g., PBMC) into hiPSCs.

Depending on desired purpose, the somatic cells may be reprogrammed via a viral or non-viral method, i.e., modifying the expression of transcription factors in the somatic cells with the aid of a viral vector or by use of a non-viral method to induce the somatic cells into hiPSCs. Specifically, in the viral method, the nucleic acid(s) encoding the transcription factors is/are introduced into the somatic cells via a viral vector; exemplary viral vectors suitable for this purpose include, but are not limited to, sendai virus, adenovirus, adeno-associated virus (AAV), retrovirus, lentivirus, and sindbis virus. In the non-viral method, the nucleic acid(s) encoding the transcription factors is/are introduced into the somatic cells via a non-viral technique, such as mRNA transfection, miRNA transfection or infection, transposons (e.g., PiggyBac and Sleeping Beauty), plasmid transfection, liposomal magnetofection, minicircle transfection, or other methods known to introduce a nucleic acid into a host cell. Alternatively, the transcription factors may be in vitro synthesized by prokaryotic or eukaryotic systems, and then administered to the somatic cells for the reprogramming purpose. Still alternatively, the hiPSCs may be produced by chemical induction, i.e., treating the somatic cells with chemicals (e.g., cAMP agonists and/or epigenetic modulators) to induce the reprogramming process.

In the step (a), the hiPSCs obtained by any method described above are genetically modified to comprise deletions in HCN genes. The HCN genes (i.e., HCN1, HCN2, HCN3 and/or HCN4 genes) may be deleted from the genome of the hiPSC by any gene-knockout technique known in the art, in which a specific gene is partially or completely removed from the genomic DNA of a cell or a model organism to prevent said specific gene from expression. Examples of gene-knockout technique include, but are not limited to, Cre and Lox recombination, clustered regularly interspaced short palindromic repeats (CRISPR) genome editing, homologous recombination, site-specific nucleases, zinc-fingers, and transcription activator-like effector nucleases (TALENs). According to one exemplary embodiment, the HCN genes are deleted from the genome of the hiPSC by CRISPR genome editing. The CRISPR mechanism and the procedures for carrying out the CRISPR technique are known by the person having ordinary skill in the art; hence, the detailed description is omitted herein, for the sake of brevity.

According to some embodiments of the present disclosure, the HCN1 and HCN4 genes are absent in the modified hiPSCs (i.e., HCN1⁻/HCN4⁻ hiPSCs). According to some preferred embodiments of the present disclosure, the HCN1, HCN2, HCN3, and HCN4 genes are absent in the modified hiPSCs (i.e., HCN1⁻/HCN2⁻/HCN3⁻/HCN4⁻ hiPSCs).

In the step (b), the HCN-deleted hiPSCs of step (a) are incubated in a differentiation medium. Depending on desired purpose, the differentiation medium may comprise suitable differentiation factors and/or molecules associated with cardiomyocyte differentiation, for example, ascorbic acid (AA), activin A, basic fibroblast growth factor (bFGF), bone morphogenetic protein 2 (BMP2), BMP4, dickkopf-1 (DKK1), an inhibitor of glycogen synthase kinase-3 (GSK-3) (e.g., CHIR99021), noggin (Ngn), stem cell factor (SCF), vascular endothelial growth factor (VEGF), an inhibitor of WNT pathway (such as IWP-1, IWP-2, IWP-4 or IWR-1), or a combination thereof, so that the HCN-knockout hiPSCs would differentiate into HCN-knockout hiPSC-CMs. According to some exemplary embodiments, the hiPSCs are incubated in a differentiation medium containing B-27™ serum free supplement, bFGF, ascorbic acid, GSK-3 inhibitor and WNT inhibitor. As could be appreciated, a skilled artisan may select suitable differentiation medium for the differentiation process in accordance with practical applications.

According to some embodiments, the cardiomyocytes derived from the HCN1⁻/HCN4⁻ hiPSCs (i.e., HCN1⁻/HCN4⁻ hiPSC-CMs) comprise a deletion of exon 3 of HCN1 gene, and a deletion of exon 6 of HCN4 gene. According to certain preferred embodiments, the HCN1⁻/HCN4⁻ hiPSC-CMs comprise a deletion from nucleotide positions 1235 to 1254 in the HCN1 gene (SEQ ID NO: 1, or NCBI Reference Sequence: NM_021072.4), and a deletion from nucleotide positions 2509 to 2528 in the HCN4 gene (SEQ ID NO: 4, or NCBI Reference Sequence: XM_033189457.1). In these embodiments, the population of HCN1⁻/HCN4⁻ hiPSC-derived CMs does not express HCN1 and HCN4.

According to some embodiments, the cardiomyocytes derived from the HCN1⁻/HCN2⁻/HCN3⁻/HCN4⁻ hiPSCs (i.e., HCN1⁻/HCN2⁻/HCN3⁻/HCN4⁻ hiPSC-CMs) comprise a deletion of exon 3 of HCN1 gene, a deletion of exon 2 of HCN2 gene, a deletion of exon 3 of HCN3 gene, and a deletion of exon 6 of HCN4 gene. According to certain preferred embodiments, the HCN1⁻/HCN2⁻/HCN3⁻/HCN4⁻ hiPSC-CMs comprise a deletion from nucleotide positions 1235 to 1254 in the HCN1 gene (SEQ ID NO: 1, or NCBI Reference Sequence: NM_021072.4), a deletion from nucleotide positions 883 to 902 in the HCN2 gene (SEQ ID NO: 2, or NCBI Reference Sequence: XM_018560576.1), a deletion from nucleotide positions 796 to 815 in the HCN3 gene (SEQ ID NO: 3, or NCBI Reference Sequence: XM_004315601.3), and a deletion from nucleotide positions 2509 to 2528 in the HCN4 gene (SEQ ID NO: 4, or NCBI Reference Sequence: XM_033189457.1). In these embodiments, the population of HCN1⁻/HCN2⁻/HCN3⁻/HCN4⁻ hiPSC-CMs does not express HCN1, HCN2, HCN3 and HCN4.

The HCN-deleted hiPSC-CMs of step (b) are then isolated from the differentiation medium by gravity or centrifugation (e.g., centrifuge at the speed of 100×g at room temperature or 4° C. for 1-10 minutes), thereby producing the isolated population of HCN-deleted hiPSC-CMs.

As could be appreciated, the present cardiomyocytes may alternatively be derived from human embryonic stem cells (hESCs). The method of producing cardiomyocytes from hESCs is quite similar to the method of producing cardiomyocytes from hiPSCs described above. Therefore, the detailed description is omitted herein, for the sake of brevity.

The second aspect of the present disclosure pertains to a pharmaceutical composition for treating cardiac disease in a subject in need of such treatment. According to certain embodiments of the present disclosure, the pharmaceutical composition comprises the HCN-deleted hiPSC-CMs produced in accordance with any embodiments of the present disclosure, and a pharmaceutically acceptable carrier.

According to various embodiments of the present disclosure, the pharmaceutical composition may be formulated into a dosage form suitable for administration through a desired route. As could be appreciated, the HCN-deleted hiPSC-CMs can be administered into any area of the heart where conduction disturbances have occurred. According to some preferred embodiments, the HCN-deleted hiPSC-CMs are administered to the left ventricle of the heart. The number of the HCN-deleted hiPSC-CMs necessary to be therapeutically effective varies with the type of disorder being treated as well as the extent of the overall damage of myocardial tissue, among other factors. A particularly suitable administration mode can be in situ application of the present HCN-deleted hiPSC-CMs or pharmaceutical composition to a cardiac tissue by, for example, direct surgical application. Another particularly suitable administration mode can be catheter injection of the present HCN-deleted hiPSC-CMs or pharmaceutical composition to a cardiac tissue. There are many known techniques that can be used to facilitate the access to the administration site. For example, the catheter injection may be used in connection with a fluoroscopy, X-ray, echocardiography, or magnetic resonance imaging guiding system. It should be noted that the above-mentioned administration routes are provided for the purpose of discussion, and the present disclosure is not limited to these administration modes. Rather, according to some embodiments, the HCN-deleted hiPSC-CMs or pharmaceutical compositions may be administered systemically. Illustrative examples of dosage forms of the pharmaceutical composition include suspensions, dispersions, solutions, ointments, pastes, powders, dressings, creams, and gels. As could be appreciated, these pharmaceutical compositions are also within the scope of the present disclosure.

Preferably, the present pharmaceutical composition is formulated into liquid forms, e.g., sterile suspensions that can be administered by intramyocardial injection. More preferably, the volume of the present pharmaceutical composition is less than 10 milliliter (mL), for example, less than 9, 8, 7, 6, 5, 4, 3, 2, 1, 0.9, 0.8, 0.7, 0.6, 0.5, 0.4, 0.3, 0.2 or 0.1 mL.

The third aspect of the present disclosure provides a method for treating a cardiac disease in a subject. The method comprises administered to the subject effective amount of the present hiPSC-CMs (i.e., HCN-deleted hiPSC-CMs) or pharmaceutical composition so as to ameliorate or alleviate the symptoms of the cardiac disease.

According to certain exemplary embodiments of the present disclosure, the effective amount of the present hiPSC-CMs for treating the cardiac disease is about 1×10⁵ to 1×10⁹ cells per transplant dose, such as 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3× 10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, or 1×10⁹ cells per transplant dose. In some exemplary embodiments, the pharmaceutical composition comprises 1×10⁵ to 1×10⁹ hiPSC-CMs so as to achieve the therapeutic purpose.

Preferably, the present hiPSC-CMs or pharmaceutical composition is intramyocardially administered to the subject. More preferably, the present hiPSC-CMs or pharmaceutical composition is administered to the left ventricle of the heart of the subject.

According to some examples of the present disclosure, the administered hiPSC-CMs are viable and functional in the subject for at least 6 months, for example, 6, 7, 8, 9, 10, 11 or 12 months, or longer.

Depending on desired purpose, the present hiPSCs and the hiPSC-CMs derived therefrom may be derived from the subject being treated/administered (i.e., autologous transplantation), another subject of the same species (i.e., allogeneic transplantation), or a subject of different species (i.e., xenogeneic transplantation). Preferably, said transplantation is autologous transplantation or allogeneic transplantation. In the case when the transplantation is allogeneic transplantation, the present hiPSCs or hiPSC-CMs are further modified to inhibit the expression of major histocompatibility complex (MHC), for example, making a deletion or mutation in MHC genes. Alternatively, the method further comprises the step of administering to the subject an immunosuppressive treatment prior to, concurrently with, or after the administration of present hiPSC-CMs, so as to suppress the immune response of the subject against the allogeneic hiPSC-CMs. The immunosuppression may be achieved by any agent and/or method known by a skilled artisan to prevent graft rejection, for example, the administration of gamma irradiation or immunosuppressant.

Depending on desired purposes, the immunosuppressant may be a glucocorticoid (e.g., prednisone, budesonide, prednisolone, dexamethasone or hydrocortisone), janus kinase inhibitor (e.g., tofacitinib), calcineurin inhibitor (e.g., cyclosporine or tacrolimus), mTOR inhibitor (e.g., sirolimus or everolimus), inhibitor of inosine monophosphate dehydrogenase (IMDH inhibitor; e.g., azathioprine, leflunomide or mycophenolate), biologics or monoclonal antibody (e.g., abatacept, adalimumab, anakinra, certolizumab, etanercept, golimumab, infliximab, ixekizumab, natalizumab, rituximab, secukinumab, tocilizumab, ustekinumab, vedolizumab, basiliximab or daclizumab), or any agent known to suppress or reduce the immune response, such as methotrexate or mercaptopurine. A clinical practitioner or a skilled artisan may determine the type of immunosuppressant and treatment regimen in accordance with the physical conditions of the subject.

According to some embodiments, the administration of the present hiPSC-CMs or pharmaceutical composition is useful in treating the cardiac disease, without causing ventricular arrhythmia in the subject. According to certain embodiments, the administration of the present hiPSC-CMs or pharmaceutical composition alleviates post-transplant ventricular arrhythmia.

Non-limiting examples of the cardiac disease treatable with the present hiPSC-CMs, pharmaceutical composition and/or method include, myocardial infarction, cardiac arrhythmia, myocardial ischemia, cardiac failure, angina pectoris, or a combination thereof. According to one exemplary embodiment, the cardiac disease is myocardial infarction.

The subject treatable by the present method is a mammal, for example, a human, mouse, rat, guinea pig, hamster, monkey, swine, dog, cat, horse, sheep, goat, cow, and rabbit. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

### Materials and Methods

Preparation of hiPSCs

In the present disclosure, the hiPSCs were prepared from human peripheral blood mononuclear cells (PBMCs) via the following steps:
(1) culturing the PBMCs in RPMI 1640 medium supplemented with 10% fetal bovine serum (FBS) and 1% penicillin/streptomycin at a 5% CO₂ incubator at 37° C.;
(2) genetically modifying the PBMCs (transduction of reprogramming factors) with sendai virus vectors to express transcription factors Oct4, Sox2, Klf4, and c-Myc;
(3) culturing the PBMCs of step (2) in STEMFLEX™ medium to support their survival and proliferation; and
(4) isolating the generated hiPSC colonies, followed by expanding the hiPSCs to establish stable cell lines; the pluripotent nature of the thus-obtained hiPSCs were determined by assessing their morphology, surface marker expression, and ability to differentiate into cells representative of the three germ layers.
   Preparation of HCN-Deleted hiPSCs

For the purpose of producing HCN1⁻/HCN4⁻ hiPSCs, CRISPR technology was used to delete HCN1 and HCN4 genes from the genome of hiPSCs. To target the coding sequences of the human HCN genes, the following CRISPR sequences were annealed and ligated into the All-In-One (AIO) vectors containing the Cas9 expression cassette, including the CRISPR guide RNAs, 5′-GTTCTTAGTACCACTACTGC-3′ (SEQ ID NO: 5) for HCN1 gene and 5′-GTGCGCATCGTGAACCTCAT-3′ (SEQ ID NO: 6) for HCN4 gene. The CRISPR transfection was performed in the order of HCN1 and HCN4. The transfected hiPSCs were cultured with STEMFLEX™ medium and then dissociated into single cells using 0.05% trypsin. The single cells were seeded in wells of a 96-well cell culture dish and expanded into colonies. Finally, real-time PCR was used to detect CRISPR editing efficiency.

The thus-produced the HCN1⁻/HCN4⁻ hiPSCs had a deletion from nucleotide positions 1235 to 1254 in the HCN1 gene (SEQ ID NO: 1) and a deletion from nucleotide positions 2509 to 2528 in the HCN4 gene (SEQ ID NO: 4).

For the purpose of producing HCN1⁻/HCN2⁻/HCN3⁻/HCN4⁻ hiPSCs, CRISPR technology was used to delete HCN1, HCN2, HCN3 and HCN4 genes from the genome of hiPSCs. To target the coding sequence of the human HCN genes, the following CRISPR sequences were annealed and ligated into the All-In-One (AIO) vectors containing the Cas9 expression cassette, including the CRISPR guide RNAs, 5′-GTTCTTAGTACCACTACTGC-3′ (SEQ ID NO: 5) for HCN1 gene, 5′-AGTACAGTTCACTCCACGAG-3′ (SEQ ID NO: 7) for HCN2 gene, 5′-ATGTCACTGGGATGGCTGTC-3′ (SEQ ID NO: 8) for HCN3 gene, and 5′-GTGCGCATCGTGAACCTCAT-3′ (SEQ ID NO: 6) for HCN4 gene. The CRISPR transfection was performed in the order of HCN1, HCN4, HCN2, and HCN3. The transfected hiPSCs were cultured with STEMFLEX™ medium and then dissociated into single cells using 0.05% trypsin. The single cells were seeded in wells of a 96-well cell culture dish and expanded into colonies. Finally, real-time PCR was used to detect CRISPR editing efficiency.

The thus-produced the HCN1⁻/HCN2⁻/HCN3⁻/HCN4⁻ hiPSCs had a deletion from nucleotide positions 1235 to 1254 in the HCN1 gene (SEQ ID NO: 1), a deletion from nucleotide positions 883 to 902 in the HCN2 gene (SEQ ID NO: 2), a deletion from nucleotide positions 796 to 815 in the HCN3 gene (SEQ ID NO: 3), and a deletion from nucleotide positions 2509 to 2528 in the HCN4 gene (SEQ ID NO: 4).

Preparation of HCN-Deleted hiPSC-CMs from HCN-Deleted hiPSCs

The HCN-deleted hiPSCs were cultured in MEM a (a modification of Minimum Essential Medium (MEM) containing non-essential amino acids, sodium pyruvate, lipoic acid, vitamin B₁₂, biotin, and ascorbic acid), supplemented with 4 ng/mL bFGF. On day −1, 1.4×10⁶ cells were seeded into a 6-well plate coated with MATRIGEL®. The culture medium was replaced with RPMI medium containing GSK-3 inhibitor (5 M CHIR99021) and B-27™ supplement minus insulin (a complete B-27™ supplement without insulin that contained biotin, DL-alpha tocopherol acetate, DL-alpha tocopherol, vitamin A, bovine serum albumin, catalase, human transferrin, superoxide dismutase, corticosterone, D-galactose, ethanolamine HCl, glutathione, L-carnitine HCl, linoleic acid, linolenic acid, progesterone, putrescine, sodium selenite and triodo-I-thyronine) on day 0. The cells were incubated at 37° C. for two days. The medium was replaced with RPMI medium containing B-27™ supplement minus insulin on day 2, followed by incubating the cells at 37° C. for 24 hours.

On day 3, the medium was changed to RPMI medium containing WNT inhibitor (5 μM IWR-1) and B-27™ supplement minus insulin. Two days later (day 5), the WNT inhibitor was removed from the culture medium (i.e., culturing the cells with RPMI medium containing B-27™ supplement minus insulin). The medium was further changed to RPMI medium containing complete B-27™ supplement (with insulin) on day 7. The medium was replaced with fresh medium every two days. On day 12, the culture medium was removed, and RPMI (no glucose) containing B-27™ supplement (with insulin) was added to the well. The medium was replaced with fresh medium every two days. On day 16, the medium was replaced with RPMI containing B-27™ supplement (with insulin) so as to obtain the HCN-deleted hiPSC-CMs.

According to the analytic results, the HCN1⁻/HCN4⁻ hiPSC-CMs did not express HCN isoforms HCN1 and HCN2, and the HCN1⁻/HCN2⁻/HCN3⁻/HCN4⁻ hiPSC-CMs did not express HCN isoforms HCN1, HCN2, HCn3 and HCN4 (data not shown).

### Animal Study

A myocardial infarction (MI) pig model (Lanyu Pig, 8 month-old, 35-45 kg) was established in the present study. One week prior to ischemia-reperfusion (I-R) surgery, an insertable cardiac monitor was implanted to the heart thereby recording the heart rhythm. Transthoracic echocardiogram (TTE) and pressure-volume (P-V) loop conductance catheterization were carried out to determine left ventricular (LV) function.

HCN1-4-deleted hiPSC-CMs (1.5×10⁹ cells/pig) and HCN-positive hiPSC-CMs (1.5×10⁹ cells/pig; serving as control group) were respectively administered to the left ventricles of the animals' hearts two weeks post I-R surgery. After four weeks, the LV function of the animals receiving HCN-deleted hiPSC-CMs (n=8) or HCN-positive hiPSC-CMs (n=6) was determined by TTE and P-V loop analysis, and the electrocardiogram (ECG) of the animals was recorded.

### Statistical Analysis

All statistical analyses were performed using software. The results were analyzed using the unpaired Student's t-test or one-way ANOVA test to compare two independent groups or more than two comparisons, respectively. For each result, all data are presented as the means±S.D. from three independent experiments. * P<0.05, ** P<0.01, *** P<0.001, and ns indicates no significance for 95% two-tail confidence intervals.

Example 1 Characterization of hiPSC-CMs

The biophysical characteristics of membrane ion currents in hiPSC-CMs were investigated in the example. First, the whole-cell configuration of the patch-clamp technique was conducted to examine the kinetic properties of sodium current (I_{Na}) in hiPSC-CMs. It is known that the magnitude of Iₕ is closely linked to the extent of pacemaker potential in different types of electrically excitable cells. Thus, RNA sequencing and quantitative polymerase chain reaction (qPCR) were used to identify whether hPSC-CMs had HCN expression. The data indicated that the expression of HCN1 and HCN4 was much higher in hiPSC-CMs than that in hiPSCs (data not shown).

These data suggested that compared to hiPSCs, the cardiomyocytes derived from hiPSCs (i.e., hiPSC-CMs) exhibited higher expression level of HCN proteins, especially HCN1 and HCN4.

### Example 2 In Vivo Study

The slope of end-systolic elastane (Ees) represents the end-systolic pressure-volume P-V relationship (ESPVR), and serves as an index of myocardial contractility. According to the TTE and P-V loop analysis, compared to the control group (i.e., the animals without I-R surgery), the VL function decreased in the animals receiving I-R surgery, in which the left ventricular ejection fraction (LVEF) declined from 74.0±3.3% to 38.5±4.4% (n=5, p<0.001) and the Ees was reduced from 6.24±1.17 to 2.41±0.74 (n=5, p<0.001) (data not shown).

The data of insertable cardiac monitor indicated that there was no ventricular arrhythmia episode before I-R surgery. However, ventricular tachycardia (VT) episodes, which could be spontaneously converted to sinus rhythm, were detected during I-R surgery (data not shown). The cycle length of these VT episodes during I-R surgery was measured. According to the results, the QRS width of VT in Lanyu pigs was ≥60 msec, while the infarcted Lanyu pigs exhibited non-sustained and sustained VT episodes of 11±9 and 59±22, respectively (data not shown). The longest duration and the fastest heart beats were 55 minutes and 375 beats per minute (BPM) (data not shown). These pigs still could survive with good activity during VT attacks.

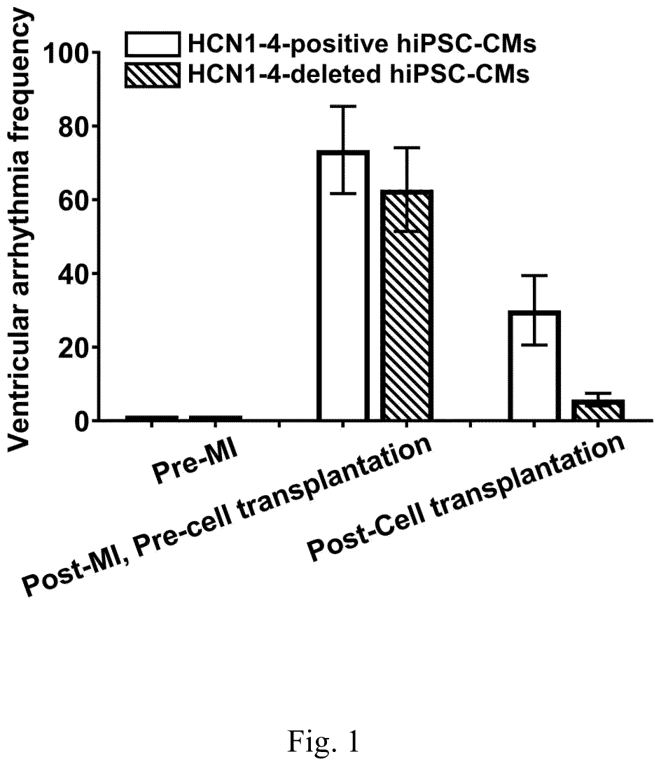

The established MI pig model was used to evaluate the effect of the present HCN-deleted hiPSC-CMs on treating MI. The data of FIG. 1 demonstrated that intramyocardial injection of HCN-deleted hiPSC-CMs alleviated post-transplant ventricular arrhythmia as compared to the control group (HCN-positive hiPSC-CMs), suggesting that intramyocardial delivery of HCN-deleted hiPSC-CMs would not increase the risk of ventricular arrhythmogenicity after intramyocardial transplantation.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An isolated population of cardiomyocytes differentiated from human induced pluripotent stem cells (hiPSCs), comprising genomic deletions in HCN1 and HCN4 genes.

2. The isolated population of cardiomyocytes of claim 1, further comprising genomic deletions in HCN2 and HCN3 genes.

3. The isolated population of cardiomyocytes of claim 1, wherein the isolated population of cardiomyocytes is produced by a method comprising,
(a) making deletions in the HCN1 and HCN4 genes of the hiPSCs; and
(b) incubating the hiPSCs of step (a) in a differentiation medium to induce differentiation of the hiPSCs into the population of cardiomyocytes.

4. The isolated population of cardiomyocytes of claim 3, further comprising making deletions in the HCN2 and HCN3 genes of the hiPSCs prior to step (b).

5. A method of producing the isolated population of cardiomyocytes of claim 1, comprising
(a) making deletions in the HCN1 and HCN4 genes of the hiPSCs; and
(b) incubating the hiPSCs of step (a) in a differentiation medium to induce differentiation of the hiPSCs into the population of cardiomyocytes.

6. The method of claim 5, further comprising making deletions in the HCN2 and HCN3 genes of the hiPSCs prior to step (b).

7. A method of treating a cardiac disease in a subject, comprising administering to the subject an effective amount of the isolated population of cardiomyocytes of claim 1 so as to ameliorate or alleviate the symptoms of the cardiac disease.

8. The method of claim 7, wherein the isolated population of cardiomyocytes is intramyocardially administered to the subject.

9. The method of claim 8, wherein the isolated population of cardiomyocytes is administered to the left ventricle of the heart of the subject.

10. The method of claim 7, wherein the cardiac disease is myocardial infarction, cardiac arrhythmia, myocardial ischemia, cardiac failure, angina pectoris, or a combination thereof.


SEQUENCE LISTING (8 sequences)

SEQ ID NO: 1 - DNA, 9933 bp, Homo sapiens
  source 1..9933: mol_type = genomic DNA
gagccgagctgagcctcgcctagctccggcagcctcagcttcagcaccagccgcaccggagcccggaccgcagccactaagggcagcggcggcggcgggagccaggcgcgcagctagcagcggcgcggtcgggaagcagcagccgccgccgccgccgccgccgccgcgacgggcagctgggctcgccggcagccggctcgggcccctaccctctcggctacgtgtctccggcgccgggcggccggcgagtctggagcccgcgccgtcgccggccgcgtcctccgggcatggaaggaggcggcaagcccaactcttcgtctaacagccgggacgatggcaacagcgtcttccccgccaaggcgtccgcgacgggcgcggggccggccgcggccgagaagcgcctgggcaccccgccggggggcggcggggccggcgcgaaggagcacggcaactccgtgtgcttcaaggtggacggcggtggcggcggtggcggcggcggcggcggcggcgaggagccggcggggggcttcgaagacgccgaggggccccggcggcagtacggcttcatgcagaggcagttcacctccatgctgcagcccggggtcaacaaattctccctccgcatgtttgggagccagaaggcggtggaaaaggagcaggaaagggttaaaactgcaggcttctggattatccacccttacagtgatttcaggttttactgggatttaataatgcttataatgatggttggaaatctagtcatcataccagttggaatcacattctttacagagcaaacaacaacaccatggattattttcaatgtggcatcagatacagttttcctattggacctgatcatgaattttaggactgggactgtcaatgaagacagttctgaaatcatcctggaccccaaagtgatcaagatgaattatttaaaaagctggtttgtggttgacttcatctcatccatcccagtggattatatctttcttattgtagaaaaaggaatggattctgaagtttacaagacagccagggcacttcgcattgtgaggtttacaaaaattctcagtctcttgcgtttattacgactttcaaggttaattagatacatacatcaatgggaagagatattccacatgacatatgatctcgccagtgcagtggtgagaatttttaatctcatcggcatgatgctgctcctgtgccactgggatggttgtcttcagttcttagtaccactactgcaggacttcccaccagattgctgggtgtctttaaatgaaatggttaatgattcttggggaaagcagtattcatacgcactcttcaaagctatgagtcacatgctgtgcattgggtatggagcccaagccccagtcagcatgtctgacctctggattaccatgctgagcatgatcgtcggggccacctgctatgccatgtttgtcggccatgccaccgctttaatccagtctctggattcttcgaggcggcagtatcaagagaagtataagcaagtggaacaatacatgtcattccataagttaccagctgatatgcgtcagaagatacatgattactatgaacacagataccaaggcaaaatctttgatgaggaaaatattctcaatgaactcaatgatcctctgagagaggagatagtcaacttcaactgtcggaaactggtggctacaatgcctttatttgctaatgcggatcctaattttgtgactgccatgctgagcaagttgagatttgaggtgtttcaacctggagattatatcatacgagaaggagccgtgggtaaaaaaatgtatttcattcaacacggtgttgctggtgtcattacaaaatccagtaaagaaatgaagctgacagatggctcttactttggagagatttgcctgctgaccaaaggacgtcgtactgccagtgttcgagctgatacatattgtcgtctttactcactttccgtggacaatttcaacgaggtcctggaggaatatccaatgatgaggagagcctttgagacagttgccattgaccgactagatcgaataggaaagaaaaattcaattcttctgcaaaagttccagaaggatctgaacactggtgttttcaacaatcaggagaacgaaatcctcaagcagattgtgaaacatgacagggagatggtgcaggcaatcgctcccatcaattatcctcaaatgacaaccctgaattccacatcgtctactacgaccccgacctcccgcatgaggacacaatctccaccggtgtacacagcgaccagcctgtctcacagcaacctgcactcccccagtcccagcacacagaccccccagccatcagccatcctgtcaccctgctcctacaccaccgcggtctgcagccctcctgtacagagccctctggccgctcgaactttccactatgcctcccccaccgcctcccagctgtcactcatgcaacagcagccgcagcagcaggtacagcagtcccagccgccgcagactcagccacagcagccgtccccgcagccacagacacctggcagctccacgccgaaaaatgaagtgcacaagagcacgcaggcgcttcacaacaccaacctgacccgggaagtcaggccactctccgcctcgcagccctcgctgccccatgaggtgtccactctgatttccagacctcatcccactgtgggcgagtccctggcctccatccctcaacccgtgacggcggtccccggaacgggccttcaggcagggggcaggagcactgtcccgcagcgcgtcaccctcttccgacagatgtcgtcgggagccatccccccgaaccgaggagtccctccagcaccccctccaccagcagctgctcttccaagagaatcttcctcagtcttaaacacagacccagacgcagaaaagccacgatttgcttcaaatttatgatccctgctgattgtcaaagcagaaagaaatactctcataaactgagactatactcagatcttattttattctatctcctgatagatccctctagcctactatgaagagatattttagacagctgtggcctacacgtgaaatgtaaaaatatatatacatatactataaaatatatatctaaattcccaagagagggtcaaaagacctgtttagcattcagtgttatatgtcttcctttctttaaatcattaaaggatttaaaatgtcgttgtaagattatttatttctaacctacttttacttaagtcctttgatatgtatatttctctattttatgaagagttcttggattcaatggaaacaaaactgattttaaaaaggcaactcaaatgaactagtaaatagcaccaatcaaaactttctttcattagctgtgtctctgcatctaaattgttaatcattaatggtggagaattaaataacaaatcccattttatagatctaaattgtatttcggtgctttcaatttcaaattaggttaaagaatgcactacttgcttggccaccgtaggagactagcattgccactgtttgttaagaatatcactaacctcaaacatgttcattgatctttcagaaagctgagggaaaattaatatttgtcttcatgtgttatcggacttttaccaagactcgatcaatgttagttgtaaataactttttcaacccaaataaaaatagctattctgtgttgtatgaaggtaaaagtcattgtttaagaatttagttttattgcttcacttcaaaacttagagttttaaattttacaaaacctgattgtgagaattattcaattgtaatgcaatggcttgaaacctacaatattattttaacctgcaatgttttatgcaaaattgtatgctcgaatctacaaattgcttgtattacaccaaaaatcgttacttttctttcttcttgccataatcaagcatctgaaaatagtccctgcatgcttctggggaaaaaaaaataggttcaaatcagtcattgtagagaaaggcttacagtatttctcgtttctagaaaagcataacaattcattaattgcctatcttataattcctataaggctgacttggatctctgacttaagtctagaagtgaggttttcacttttatttaatattattgctattattatttaaataattatttgtaaaccacagcttgatttggagttaagtgttgtctgtgtcttcactgtagctggtaatttactatggtattaatttaaagaaataaattcaatacagcatcacgtctgtttgggaacacacattttgctctatgtatattgtaactaaatggttagtaaactttaaggcatgtggtgactaacgtagcccttagataggaggtttgtcatttttaatgtgttgaaaatagtacagacatggtgacagaaagcaggggtaaagttgaacgtctatgagcattgtctgtctgttttcttaactctttacatgatctgtgagcacctgcttcttctcaaagaactcccttagtaatcatttgtgcctaatgcatcccacacatgtggagtatgactggaacacaattgaaagattcctaacaactggctgcggtaaataacccaagttaagagcatgacttaaagtggaggtcttcagtatctctggcactggcaatacatttaagaacatacagagatcacacattatgtgggacagggcctgcagatagacttgaggacagaaaacaaaaacaaaagcagaaactctaaagtggatccttgctgcctgttatgccttttgtggatataatacctataataccaaaatgagttgttatgtttaatttcaaacaaatcccaactggtgtggaagtaattaactgctagtcgatgtaatgtaatcatcaatatgctggtgacatatgatggcatgtcaggaatactgttgtaagaaagggattgttcagtctacctagaacctatgggaaagctgcaagaccatttacatatcagtacaaaactcctttattttattaaaatatgagaattaacttcaatttattaagattacgtattggtcctttaaattattaaaggtttacatttgataggattaatggatttggtagtgaaatatttttatatatataaatttttggttttttattttgagcactattgggaaacataagcaaaattgaaatgcatggtctttcagtgcaaccatacaatttttattcactttgtaatagaatggacaaaaaaactaaggtttatatattaaactaataaaaaggtacagggtaaattatatacatatatgtatgaatgtatatatacttagattaagaaaaaaaaatgactcacattcctgtaatataaaggtccattgctagttgaaaagtgacctcttttctctgtacatagtgaagcccgtatgtatggagttttaatctgtacagaaagaaatgtattattcattggtctttctgctgctggaaagataatagaatagctgattcttacaacaactgtatgatcagggatgagttagagtatttcttttgtctttgctcaaagccatacatatataaatatatataaagattattaataaattcaacaaaaaataaactacaatcatgaatattttattttatcaatatatgctaatcaaatgttttattttattaatgagtacagctttgcctctatttttacatcataaaatttccaagagcttatacttgataatcacataaaatcaattatattctataattctggaatgagaggactttaaatagaacatggatttttcaaccacttctttgtttacctttttcgtatagtaaaatatgagcttattttcttttttgctctgtgcctttttagaatgtggttgttctccaacttcaaaacacaaccattttgtgtaattattggatatacagatgagaaataattttcctgtaataatggtaaataattgtcctgtaatatatttatacaaacctattttgttataatagttctatgacatacctattttgttataatagttttataacatacctattttgttataatagggccttgggctctattataacaaaacaggtttatataaatacattcatatttatagccttaaacaaaataggtttgtatagtaggcccaaggccctattataacaaaataggtttgtataaatatattcatatttatagccttgggccctgcaaagttatttggagagtgatagaacctccccacacaagtgggttttaaatgatacctaaaagcctgagatagagtttatcataatgagaataagtagtgaagcattcctgaaaacaaggaggataaatagtagcaatggcaatatattctattcaccaaaaaagtagcttgtctccaggtctggatgacctcttcataagtgaacaattccctgatgaatgctgttccactttaaagagatggttattttgcaatttataaacttaactgagcatactattttcaggacactacaaagtacatagtcaagttattaccgtcattcctataggacagagaatgatttaaacaatatgacatgtttaaaattttagatgatttatttcaaatcctgaattatgtctacttcaaattatgagcaatctctattatctttcaggactatttaaaaaatctccatacctcctctttcagcttaggtttttatatttgttaattttactatgttactttgcttctatggccctcacttgcactgtttataatgttaacctgtttgaatgagatatctctccattatatctttatctttttagaacatgtgccataaaacccatgtggttattttgttcatatgcactacaactagcaaacttttattttatgttcacctactttaaaaagtactcatactgtaccatgggaaagaaacatgggccttatagaatggaatgctgagctattttgagtcctgaaaacaatggccaaaaatttaaggaactgtggttgctgcatgcaccaaattgtagcacatacaaataaataaaatacagttctataactgtattcaatatggttattttcaaggaaaaataggatagaaattttctttgaaagatttgctattgtacttagggaagtacacctgctattacccctcaaaagcaatgactttcttaagcaatttcaatttttttcctttttaacaaccctgcacttgcacaaccctgcatctcttgtctcaccctaatgctgaccctgcttccaattcaggctttgacagggacaaatggagattttaaggttttttaatgagagaagccttgctgacactccccgcatcccaccttgaaaattatttaaagatgatcttaaaagaacctacagataatgctctgaatttgtttacaaggtccttgatgtggagagaatgagtaatacattaaatgctggggaaataattatcatttagtgaaaaattaagcatgcttttctaaaatgtggatgtttaacagagcacagctgattgggaacactggcagttcaatgcagctggagtgtctcacagggtgaagaggaacccagatcaagtgggaaatagggatttcaaaaaccttctgaaaggcttttaacatcagggcaaaggttttacagtgttgtactttatattcttaatttaagcttatcaacggggaggattcacattgaaatgtctgttttgctaatgtaacaaggaataaaataatttatgacacagcaaaaactgggggctatttacaaccccaccctgccttgtgaggatgctgggtcctaaggaaggtcctaggtttgctaaatagagtaatgggaataggaaactgaaagtttacttgctttgcgtaagataatatattgcggcagggaaggggtacaagaagataagagctagaacctgtgagaagggaaagaagtgataaaataattttggctaaaataaagcaagttctcaggccaggcgtggtggctcatgcctgtaatcctagcactttgggaggccaaagtgggaggaatgcttgaggccaggaattggagaccaaccagggcagcatgtcaagacctggtctctacaaggaaaaaaaatttttttaattgaccagtatatacttacatacctgcagtgtttagaaaacccgactataggtggtgtattcctgtagtcctagttactcagaaagttaaggcaggaggatcgcttgaacttaggaggtcaaggctgcagtgagccctgttcgttccactgcactccagcttgggggacagagcaaaaccctttctcaagaaaaacccaaacaaacaaaaaagtgaagccagttctttaatttctattggagatatcacacacgctccaaagagtggcaaaggtgaacattctttggagctattatatttctaatgtaacatcaatggccatttactcccaaagggaaacatctttttttttttttttttttaagatggagtctggctctgtcacccaggtgggagtgcaatggcaggatcttggcccactgcaacatcagcctcccaggtttaagcaattctcctgcatcagcctccagagtagctgggattacaggtgcccgccaccatggctagctaaatttttgtaatttttagtagagacagggttttaccatgttggccaggccggtctcgaacccctaacctcaagtgatccgcccgcctcaccctccagaagtgctaggattacaggcctgagccaccgcacccggccctgctatttttttctatgtagttgtttcagtaataccatatgagcagagaatactattgattcattcaattgaagaaatctgaataaattagtaaatataataattcttcaaaagaggaccactttatgtttccaatagattatataattgaattcaggtaacgtagagttattcagacttttttttttaaataacggaattttgcaaaaaaacaggttgcatcgttttctacagagaatattcagatgtgtagaggcaaagagactttcttgaaaggtttttatttcctgttaaatttgtgaggaaaactcctgagaaaggaaagatttacttttgaaggaagaaagaacataacccaaagaaatttgaaagattcaaaaagagttttccaaatgtattctcatttctttctcatatttgttcaaggtcttagtttccaagcattcaaaattaaatcttgtttgcctaactcttctaacatctattaccaacctaacatattacaattctgtttaaaatgtgcagttctaaatgttcttgcgtgcctcactgtggaaagtgtggtgcatggaactgcagcgtcagtatcacccagaagtataattacaagaagtgcagagtccttggccttaccctgtacctacagaagtccgcttttgggcaagatccccagatgattcatatgtgtattaatattgaaaaaattggaactagtaagtcctagaggaggtaaattttaagggctttttaagggaacttccaagtgtgaagacatttctggagatgacaagtcaacatagcatgggatcttggctagacaaggaaagcgagagaacattaaataaacacaagcaacgacaaaccacctatgttataattttatcctggaccatccaaagtttactcttggtcacgctgtctaacccagcctgcctcctggcacttgctagttagaaccagggaagagaaaactaatatgtgttaggtattataatttccttatgtcacataaattttggtgccatagctagcaatgtttcttaatatcacatcagtacaatgtgtccattgccttctgtaacatttttaatgctatcaataatacattgacattataaaaataaatgaatgtggtcaata

SEQ ID NO: 2 - DNA, 3418 bp, Homo sapiens
  source 1..3418: mol_type = genomic DNA
gaacggggagtgcaggcgggagcttttcgagacgccgccccgggaacacaaggtgtccttctccaccgaacctccgcgggggtcctccccggggaaagacaccgaggatggcgaggccggagaaggggatcctcggtccggctttatgcagcgccagctttctgccatgcttcaacccggggtcaacaagttctcccttcggatgttcgggagcgagaaagcggtggagagggagcaggagagggtgaaatccgcgggggcctggatcatacacccgtacagtgacttcaggttttattgggatttcacaatgctgctctttatggttgggaacctaattatcattcctgtgggaatcactttcttcaaagaggaaactaccataccatggatcatatttaatgtggtgtcagacaccttctttcttatggacttggttctgaacttccgaactggtatcattattgaggacaacactgagattatcttggatccacagaagatcaagcgcaaatatctgcgcacatggttcgtggtggactttgtgtcctccatccctgtggattacatatttctcattgtagaaaagggcattgactctgaagtttataagacggcccgtgctttaagaattgtgaggtttaccaagatcctcagcctcctgaggttacttcgactttccagactcattcggtatattcatcaatgggaagagatcttccacatgacctatgacctggccagcgcagtgatgagaatttttaacctgattgccatgatgctactgctttgtcactgggatggatgtcttcagttcctggttccaatgctccaagacttccccagtaattgttgggtttccatcaacaagatggtgaatgactcgtggagtgaactgtactccttcgccctgttcaaggccatgagccacatgctgtgcattggttacgggcgccaggccccagaaagcatgactgatatctggcttacaatgttgagcatgatagtgggcgctacatgctacgccatgttcattggacatgccacagctctcatccagtcactggactcatcccgcagacagtaccaagaaaagtacaagcaagttgagcagtacatgtcctttcataagctgcctgccgattttcgtcaaaggcttcatgactattacgaacatcgttaccaggggaagatgtttgatgaagacagtatccttgaagagctaaatgagcctttgagagaggaaatagtgaacttcaactgccgcaagctggtagcatcaatgccactctttgccaatgctgatccctgcttcgttacggcaatgctgaccaaactaaaatttgaggtgttccagcccacggattacatcattcgggagggaaccatcggcaagaagatgtacttcatccagcatggtgttgtcagcgtacttaccaagggcaacaaggaggtgaagctctcggatggctcctactttggagagatctgcctgctgacacgggggcgacggactgccagtgtccgcgccgacacatactgccgtctctactcactctctgtggataatttcaatgaggtgttggaggaatacccaatgatgcggcgtgcatttgagacggttgccatagacagactggatagaatagggaagaagaattccatcctcatgcacaaggttcaacatgacttgaactcaggtgtcttcaacaaccaggagaatgaaataatacaagagattgtcaagtatgatcgggagatggtgcagcaagctgagctccatcaacaccaactaagcggtgggatgtatgccacatgcaatcctccctctcctcaaccccaggtcccatctgccatagcaactttgcagcaagctgtggccatgagcttttgtcccccaatggccagcccactagtgggttccccgcgtataatgaggcgactccaatatgcccaaggagggcctggagctttttctatatccccagctcttttccagccatccctgcagctcttgcagcatcatccacccccccagcaggttccacgtaggccatccactgctgtgcccagtccccccaaccaatcaccacgagtgtctcagaactttgcctacccagccaggcctcgggcaggctcacaactttctcttacccaatctgggggttctggttccccacaacgtctgtctgtccacaagagcactcattctctgcactctggcacactcacgcgtgagtctcgacccctttcagcctcacagccctcactgcctcagggggcgtcacagctccgcagtccaccagctcctcctgctacaactggcagcttaacctctccggtggccaaaggaggtttgggaccacaggctggtccaagagtacctgcccctgcaagacttgcgctctcccatcagttgtcctcttctggtacagttggtggtgtctcggactctgcagtagggacatcgttaggaggcaggaaagattctgtggttagtgccccagattctgaccctgttaggggacggctgtctgcaaacttgtgactgatgggtgtacacgttccctcactcaaatgggtaggtaatggatggtgcacagttgttattaagatagaaaatataatgtgccctcctagccatctctgtgccactttattaataactctacttgtttaaagattagtagccatacctttcctctggagtgggggtattaatatgtggaagttacaagagaactagcagttagatcagtaaatgccacttcagaaagcaatccgccacataactcctccagtgtttacaaaagtaggaaaatgcagcactgttcaatgtcacagatctacctagagctctgcattgaaatgaagaagagtgcaaatcttctgagggtctaaggcagctggtctgctgtcagaaggaaagagactgctggtatttactactgagtttttttttagttatttaaattatatatatatatatacacacatatcaaaaactctttcgctggtagatcctcactctgaattcgcaaattgaaaaagctacaacgaatattacttatgcccaaagcaagccaatttcgttatgcaaactgcaggcagtttgacctagctattggtcctttccaggactttaactacccagtgacagtgtcacctatggatgtcatgataagcacctatatttatataaccatgcatacatttattttagtttgtaatcatgttttatttaggaggaggtaaatttttttgtgttatgtcaccatttgtgcccccatttcctttcagtgagcaaatcaccaactgaatatagggattcaaattaggcactgcaagtcccagtgacacactacatactattaattgtattgacagcagtaacactgcatgtggctgttgtaaaaaa

SEQ ID NO: 3 - DNA, 3732 bp, Homo sapiens
  source 1..3732: mol_type = genomic DNA
catggaggcggagcagaggccgacgactagggccagcgatggggcgacccctgggctggagagggcgcctcctgctgccccggcgtctacaaccacggcctcggatccgattcctgggtccaggcccgggaccgggcccgagcccaagaggaggcagctcgggacgctgctccagcccacggtcaacaagttctcccttcgcgtgttcggcagccacaaagcagtggaaatcgagcaggagcgggtcaagtcagcgggggcctggatcatccacccctacagcgacttccggttttactgggacctgatcatgctcctgctgatggtggggaacctcattgtgctacctgtgggcatcaccttcttcaaggaggagaactccccaccttggatcgtcttcaacgtcctctctgacactttcttcctgatggatctggtgctcaacttccgcacaggcatcgtggtagaggagggtgctgagatcctgctggcaccgcgggccatccgcacacgctacctgcgcacctggttcctggttgatcttatctcctctattcctgtggattacattttcctggtggtggagctggagccacgactggacaccgaggtctacaaaacagctcgggccctgcgcatcgtgcgtttcaccaagatcctcagcctgctgcggctgctccgcctttcccgcctcatccgctacatacaccagtgggaggagatctttcacatgacctatgacctggccagtgctgtggttcgcatcttcaacctcatcggaatgatgctgctgctatgtcactgggatggctgtctgcagttcctggtccccatgctgcaggacttccctcccgactgctgggtctccatcaaccatatggtgaactactcgtggggccgccagtattcccacgccctgttcaaggccatgagccacatgctctgcattggctacgggcagcaggcacctgtgggcatgcccgacgtctggctcaccatgctcagcatgatcgtgggtgccacgtgctacgccatgttcatcggccacgccactgccctcatccagtccctggactcttctcggcgtcagtaccaggagaagtacaagcaggtggagcagtatatgtccttccacaagctgccagctgacacacggcagcgcatccatgagtactatgagcaccgctaccagggcaagatgtttgacgaggaaagcatcctaggagagctgagcgaaccacttcgggaggagatcattaacttcacctgccggggcctcgtggcccacatgccactgttcgcccacgccgaccccagcttcgtcacggccgtgctcaccaagctacgctttgaggtcttccagccgggggacctcgtggtgcgtgagggctccgtgggcaggaagatgtacttcatccagcatgggctgctcagtgtgctggcacgtggcgcccgggacacccgcctcactgatggatcatactttggggaaatctgtctgctgactcggggccgacgcacagccagtgtgcgtgctgacacctactgccgcctctactcgctcagcgtggaccatttcaacgccgtgctcgaggagttccccatgatgcgccgggccttcgagacagtggccatggatcggctgcgccgcattggcaagaagaattccatactgcagcggaagcgctctgagccaagtccaggcagcagtgggggcatcatggagcagcacttggtgcaacacgacagggatatggctcggggtgttcggggcctggccccaggcacaggagctcggctcagcggaaagccggtgctgtgggagccactagtgcacgcacccctgcaagcagccgccgtgacctccaatgtggccattgccctgacgcaccagcggggccctctccccctctcccctgactctccagtcaccctccttgctcgctctgctcgaagatcagcaggagcaggctccccagcctccccacttgtccctgtccgagccggccctctgctggcccggggaccctgggcatccacttcccgcctgccggccccacctgcccgaaccctccatgccagcctatcccgggctgggcgctcccaggtgtcactgctggggcctcccccaggaggaggtggacggcgactaggacctcggggccgcccactctcagcctcccagccctctctgcctcagcgggcagcaggtgatggctctcctgggtgtaagggctcaggaggtgaacgcctgcccccctcagggctcctggctaagcctccagggacagcccagccccccaggccatcagtgcctgagccagccactccccggggcccccaactctcttccaacatgtgaaacctttgggtaagctcttgggtgccatagcatgtgccagagggcagacgcctcttggggacaccaaggggacctgaaacactgctctatgggagtatctccccttagtaccaagaagatggtctctgttctcagctcaggcgccttgcaacctgacatcctcctaatccattcacccattcatcaaagatacttagcgcctaccatgttcaaggcactgtaccagacgctgtatgtctccactgccaagtagaagtgactccaagccctctgatgagggtattcccctagccatggtcctgccaggtgcaggcctgggcccatgatcccatcttactaagcacaagtacttgccaccaccatcactgccaagtaactagatgtctctgttttcctgcccatgaccttgcaggttctgcctggcatggttatcttcctgtccgctagcatagctgggcactgccaattacctgtccccccgctgctgtcaacaaatgtcttgggtccctggtgtgggcatccagctttactgccatgcgtggaccctccctgtctataccccccgagtgggaacacacatctcctgacaagttccttgcactctctgtctctgtgatagaactgcctttttgtaaactgagagccacctcctccctatgcccctgtcccagggacaacccccttgaagctaagggattgttgacacccccttatttagtgtgccagcgtagattccccccaggttgggggcaagtgaatccttagatggaattttttctttccttgctcccttccttatttattcaatcatttaataattgtatttattcactcatttgctagttttatttattatcaattcattttatttgtccattcatttatccatctctcccatcccctcctggtagggagacaggaatgggcagagcccctccatgccagctcttgtggtccttgcgcaactcccgtcagtggcaattcttgagccctctccctaactaggtaggagcaggaaggccacctgagagggagaggaggactgttcttatttttaggctttttttcctttctactgagtttgctgctggtgcaggagtaaggggagggcccgaggtaaccaagcctggggaagggcaggctagccagtacctctgccttctcagggacaagagaagccccctatcccacccctctgtccccacacctactctacagcatcagagaatgaggggccaggaccctaaatctccttttctcctgggtgggcagttctggggttctgggtgtgtgggagaggttttatattgcttccaaacacctgggtttaaaaagaaaatagagacacttgttttggctcctggcttgtg

SEQ ID NO: 4 - DNA, 7476 bp, Homo sapiens
  source 1..7476: mol_type = genomic DNA
gtcccacccgcgcgccctgtgcgtgtctccctgtttccccacgccctccttgcctggctgctgcgaggctttgcaggggaaagactggcgcggggttcgccccctgcccatgtcacaggaaaaccctgggagcctccccgacgcggcgcatttccggaaacccctccccaagcgggttgcgggaaggccgggactctgcatccagaagacccggtcgcctcccaaaaggaagagtcggagacccagctgaggggcaaacatgccagggaaaggcgagcccagagcttggtgatggagaatttgggaagccaccccgcccccttcaatcttgggatggggaattcgcaactgaagccggagctctagacttggggcgcgctcccagcttggcccaagaaagagatttaagggcgcagcagtatggacacctctcaccccggctccgaaggtctagcgagggtctaacccgggcccctcgccaggcccgccccccgccccccgccccctcccagcccccggcccgtgcgccgctgcccctttaagaagcccaggtaggcaggccgggctgcgggagccgctcctatggcaacccgcgagctgcggcggcttcatgaatattccggggcgcgggagcccgagcgcggccggagggcgctccgggggaggcggccgctgatgtaagcccggcgggtcgctgggctccgctcggctgcggcgggagccccgggacgggccagaggggccgggccagaggaggcgagacgaggtcgcagggggccagccacaaagtccgggcggcgagacagacggacggccagccctcccgcgggacgcacgcccgggacccgcgcgggccgtgcgctctgcgctccggagcggttccccgagcgccgcggccgcagagcctctccagccggcgcccattgttccccgcgggggcggggcgcctggagccgggccgcgcgccgcgcccctgaacgccagagggagggagggagggagggaggcaaggagggagcgcggagtccgagcgcccagcccgggaggaggcgcagcgcggcgagcccggggagtcggagcgcaactgggagcctcccagcggcgcacagcctgcccgagcatgggcgcctgaggcggccccacgccggcggcgaaggacgagtccccacgggcggactgactggcgggcggacctggagcccgtccgcggcgccgcgcccctatccccggcccggtccggccccggcccctggcgccatggacaagctgccaccgtccatgcgcaagcggctctacagtctcccgcagcaggtgggggccaaggcgtggatcatggacgaggaagaggacgccgaggaggagggggccgggggccgccaagaccccagccgcaggagcatccggctgcggccactgccctcgccctcgccctcgccctcggcggccgcgggtggtacggagtcccggagctcggccctcggggcagcggacagcgaagggccggcccgcggcgcgggcaagtccagcacgaacggcgactgcaggcgcttccgcgggagcctggcctcgctgggcagccggggcggcggcggcagcggcacggggagcggcagcagtcacggacacctgcatgactccgcggaggagcggcggctcatcaccgagggcgacgcgtcccccggcgaggacaggacgcccccaggcctggcggccgagcccgagcgccccggcgcctcagcgcagcccgcagcctcgccgccgccgccccagcagccaccgcagccgacctcctcctcctgcgagcagccctcagcggacaccgctatcaaagtggagggaggcgcggccaccggcgaccagatccttccggaggccgaggtgcgcttgggccagtccggtttcatgcagcgccagttcggggccatgctccaacccggggtgaacaaattctccctaaggatgttcggcagccagaaagccgtggaacgcgagcaggagagggtcaagtcggccggattttggattatccacccctacagtgacttcagattttactgggacctgaccatgctgctgctgatggtgggaaacctgatcatcattcctgtgggcatcaccttcttcaaggatgagaacaccacaccctggattgtcttcaatgtggtgtcagacacattcttcctcatcgacttggtcctcaacttccgcacagggatcgtggtggaggacaacacagagatcatcctggacccgcagcggattaaaatgaagtacctgaaaagctggttcatggtagatttcatttcctccatccccgtggactacatcttcctcattgtggagacacgcattgactcagaggtctacaagactgcccgggccctgcgcatcgtccgcttcacgaagatcctcagcctcttacgcctattacgcctctcccgcctcattcgatatattcaccagtgggaagagatcttccacatgacctacgacctggccagcgccgtggtgcgcatcgtgaacctcatcggcatgatgctcctgctctgccactgggatggctgcctgcagttcctggtacccatgctgcaggacttccctgatgactgctgggtgtccatcaacaacatggtgaacaactcctgggggaagcagtactcctacgcgctcttcaaggccatgagccacatgctgtgcatcggctatggacggcaggcgcccgtgggcatgtccgacgtctggctcaccatgctcagcatgatcgtgggcgccacctgctacgccatgttcattggccacgccactgccctcatccagtccctggactcctcccggcgccagtaccaggaaaagtataagcaggtggagcagtacatgtccttccacaagctcccgcccgacacccggcagcgcatccacgactactacgagcaccgctaccagggcaagatgttcgatgaggagagcatcctgggcgagctgagcgagcccctgcgggaggagatcatcaactttaactgccggaaactggtggcctccatgccactgtttgccaacgcggaccccaactttgtgacgtccatgctgaccaagctgcgtttcgaggtcttccagcctggggactacatcatccgggaaggcaccatcggcaagaagatgtacttcattcagcacggcgtggtcagcgtgctcaccaagggcaacaaggagaccaagctggccgacggctcctactttggagagatctgcctgctgacacggggccggcgcacagccagcgtgagggccgacacctactgccgcctctactcgctgagcgtggacaacttcaatgaggtgctggaggagtaccccatgatgcgaagggcctttgagactgtggcgctggaccgcctggaccgcatcggcaagaagaactccatcctcctccacaaagtccagcacgacctcaactcgggcgtcttcaactaccaggagaacgagatcatccagcagattgtgcagcatgaccgggagatggcgcactgcgcacaccgcgtccaggccgccgcctctgccaccccgacccccacacctgtcatctggaccccgctgatacaggcaccactgcaggctgccgctgccaccacttctgtggccatagccctcacccaccaccctcgcctgcctgctgccatcttccgccctcccccaggatctgggctgggcaacctcggtgccgggcagacaccaaggcacctgaaacggctgcagtccctgatcccttctgcgctgggctccgcctcacccgccagcagcccatcccaggtagacacgccgtcttcatcctccttccacatccaacagctggctggattctctgcccccgccggactgagcccactcctgccctcatcgagctcctccccaccccccggggcctgtggctccccctcggctcccacaccatcagctggcacagccgccaccaccacagccgggtttggccacttccacaaggcactgggtggctccctgtcctcctccgactctcccctgctcaccccgctgcagccaggtgcccgctccccgcaggctgcccaaccacctcccgcaccacccggggcccggggaggcctgggactcccggagcacttcctgccacccccaccctcatccagatccccatcatctagccctgggcagctgggccagcctcccggggagttgtccctaggtctagctgctggcccaccgagcacgccagagacacccccacggcagcctgagccgccatcccttgtggcaggggcctctgggggggcttcccctgtaggctttactccccgaggaggtctcagcccccctggccacagcccaggccccccaagaaccttcccaagtgccccgccccgggcctctggctcccacggatccttgcttctgccacctgcatccagccccccaccaccccaggtcccccagcgcaggggcacgccccccctcacccccggccgcctcacccaggacctcaagctcatctctgcatctcagccagccctgcctcaagacggggcacagactctccgcagagcctccccgcactcctcaggggagtccatggctgccttcccgctcttccccagggctgggggtggcagcgggggcagtgggagcagcaggggccttggtccccctgggaggccctatggtgccatccctggccagcacgtcactctgcctcggaagacatcctcaggttctttgccaccccctctgtctttgtttggggcaagagccacctcttctggggggccccctctgactgctggaccccagagggaacctggggccaggcctgagccagtgtgctccaaactgccgtccaatctatgagctgggcccttccctcttctttcttcttttctctcccttccttcttccttcaggtttaactgtgattaggagataaaccaataacagtaataattatttaaaaaaccacacacaccagaaaatcaaaagacagcagaaaataaccaggtattcttagagctatagatttttggtcacttgcttttatagactattttaatactcagcactagagggagggagggggagggaggagggagcaggcaggtcccaaatgcaaaagccagagaaaggcagatggggtctcggggctgggcaggggtcggggtggccagtgttgggggttcttagagcagatgtgtcattgtgttcatttagagaaacagctgccatcagccccttagctgtaacttggagctccactctgcccccaggaaggggctgccctggggcgtgccctggggagcctcagatgcctgcgaccttgggagaaaagggccagggccctgagggcctagcatttttttctactgtaaacgtagcaagatctgtatatgaatatatgtatatgtatgtaagatgtgtatatgtatagctatgtagcgctctgtagagccatgtagatagccactcacatgtgcgcacacgtgtgcagtctagtttattcctatgttgccaggatgcccgggtcaccttacacccagcaacccaccttggcccgcaggctgtgcactgcatggtctcgggatgttctctctccagtcctcagggaagaggaccccaggacttcgcagcaggccccctctctcgtctcaaatccagtcccagcctgacctctcaccacacggaagtggaagactcctttcctagggcctcaggcacaccctgccacctctggggccatcagtttgcccatctgtacagcgggaggtgaggggaacttctgtgtattgagtctgctctgtgccaagcactggtttcgctctttacacatgttcactccttcagtttcacaaagaccatggggtgggcactttcattctcccgatttagcagatgaagaaacagttctgggtaatttctccaccatcatgtaactcggagtggcagagtggggactgatttgaggttcaaatccacgcctgcttgaggcccaagtctgtgttccttccatcagaaaactgttgagggagtctgaggtagatggtccccaagcgtggtgcagaaggaagacaccagattttggcagcagtcaggcctgggtttgaatcccagccctaccacttcttagctgtatgatcttgggcaagttatctgacttttctgtcacctcatttgtaaaatggaaatcattatggtaccgcctcacaaggacctatgaggaccagatgcgaaaaatctgtatgtgaaatgcccagcccagcgcctggcacataccatggtaggtgctcaatcaatcacatttcttctgcccctcatatgcccagcctattgctccagcaaactatgtgagagcccagggagctttggctgagggctccaagacttaaaatctcaggactcaggaggtggctgggcctccctaagggcccaaggaaggtgtgtggccagaggtgggtgggagccaggccttgagatgtgggaagacttcaacagggagagagggagggaatggtgggtgggatggagtatatagcggagacattcctgaggtggatgtggagtggtggatcagggctttgggagggaatccgcaggctgaggggtcagagggatggccttgggtgataggctaagggattgactgggcttggtcctggcaactaggagccataagcaggttccagattgcaggaacgagaaagcagctcagatgcctttggaggcaccatcctccctcctcccagatgggatcctgccagaggcaaagtcaggggtctgcccctgcctatagggccagggcaggtatagctgcaatccccaaataatgagatgggctggccccacattatccgtccagaacattccatgcttcaagccagaatgttggcaagatcgagttttgccttgagctatcctgggatgtgggaaaaaccgatttctccatagatgggctgcagggagtgggaggcggtactccaggagagaagtgggtgaaggttcctgggatcttaggtaaagactagatgccgcctggtactggtctgtactgtgctggctcaggagttctgagaactggaaggacttagcctcaacctgagttctgcacacaccccttccccttaaggaaggcagctctgagaggcagcaggacttgatccaaacccacagtcttgtcctggaggcagcaggggtgaaggtggagggtccagggccgtgaggaggctgcttgccatcagatcctggcctaaccaccctcttctgtacttacacacacatgtattttataatagctctgacccaacctggccactctgcagagactgggacagccaggtgcaggcagtgggccctcccacacccagtcacctacaaggaattttcaaatcttccacttttaaaaaagaaaccggtaaatgcgccgtattgtatattttatttaaataaaaaaaaattccagca

SEQ ID NO: 5 - DNA, 20 bp, synthetic construct
  source 1..20: mol_type = other DNA
gttcttagtaccactactgc

SEQ ID NO: 6 - DNA, 20 bp, synthetic construct
  source 1..20: mol_type = other DNA
gtgcgcatcgtgaacctcat

SEQ ID NO: 7 - DNA, 20 bp, synthetic construct
  source 1..20: mol_type = other DNA
agtacagttcactccacgag

SEQ ID NO: 8 - DNA, 20 bp, synthetic construct
  source 1..20: mol_type = other DNA
atgtcactgggatggctgtc